(19) United States
(12) Patent Application Publication
(10) Pub. No.: US 2011/0136243 A1
(43) Pub. Date: Jun. 9, 2011

(54) HIGH THROUGHPUT METHOD FOR ANALYZING THE FATTY ACID COMPOSITION IN PLASMA PHOSPHOGLYCERIDES

(75) Inventors: Claudia Glaser, Munich (DE); Hans Demmelmair, Munich (DE); Berthold Koletzko, Munich (DE)

(73) Assignee: LUDWIG-MAXIMILIANS-UNIVERSITAT, Munich (DE)

(21) Appl. No.: 12/997,107

(22) PCT Filed: Jun. 5, 2009
(86) PCT No.: PCT/EP2009/004043

§ 371 Date: Feb. 9, 2011

(30) Foreign Application Priority Data
Jun. 10, 2008 (EP) 08010504.2
Sep. 8, 2008 (DE) 102008046227.6

(51) Int. Cl. G01N 33/92

(52) U.S. Cl. 436/71

(57) ABSTRACT

A method is described for determining the fatty acid composition of phosphoglycerides. In said method, methanol is added to a sample containing phosphoglycerides, the combination is mixed, precipitated material is separated from the methanol phase, an alkali alkoxide is added to the methanol phase as a base in order to catalyze a transesterification process, and the produced methyl esters are extracted from the solution obtained following the transesterification process and are gas-chromatographically separated. A test kit that is suitable for carrying out said method is also disclosed.

The present invention relates to a method of detecting the fatty acid composition of phosphoglycerides, and to a kit suitable for this purpose.

Plasma contains fatty acids either in the free state or bound to lipids, which in turn can combine with proteins to form lipoproteins. The fatty acids can be bound inter alia to phospholipids, of which phosphoglycerides are a subfraction, cholesteryl esters and triglycerides. It has now been established that the fatty acid composition of blood, and especially the fatty acid composition of phospholipids, correlates with the fatty acid composition of cells and membranes. In addition, lipids play an important role in organs and cells. Thus the nervous system has a high lipid content.

It is further known that there is a relationship between the fatty acid composition and the proportion of specific fatty acids, i.e. the fatty acid distribution in cells and blood, and a very wide variety of diseases and conditions. Thus, for example, in Am. J. Clin. Nutr., 2008, 87: el. 70 to 80, Vaisman et al. report that the fatty acid composition of blood correlates with syndromes such as hyperactivity and attention deficiency syndrome in children, and that diet can influence attention deficiency syndrome in children by changing the pattern of fatty acids, e.g. the proportion of polyunsaturated fatty acids. This change in the pattern of fatty acids makes it possible to eliminate disorders, although these changes have to be monitored. This requires reliable methods of detection that can be carried out with small amounts of sample.

Of particular interest in this connection is the proportion of polyunsaturated fatty acids, e.g. long-chain polyunsaturated n-3 fatty acids (LC-PUFA), which is linked to the mental development of children and the elderly. The “FA status” plays an important role in many clinical trials and tests relating to nutrition. The FA concentrations could be valuable biological markers for the quality and metabolic pathways of food. It would therefore be desirable to have a reliable, high-throughput method that is easy to carry out.

Furthermore, in Human Molecular Genetics, 2006, vol. 15, no. 11, 1745 to 1756, L. Schaeffer et al. report that the fatty acid composition of membranes plays an important role in cellular processes and is associated with the etiology of various complex diseases. As it has also been found that the fatty acid composition of phospholipids in plasma correlates well with the fatty acid composition of tissue, and a relationship has been established between the fatty acid status of tissue and the mental development of children with diseases such as heart diseases, cancer and autoimmune diseases, the detection of fatty acid composition becomes increasingly important. Above all, a rapid, routine determination of fatty acid status would be valuable as a medical marker as well as a nutritional physiological marker.

Because of the importance of fatty acid composition, methods of determining it have already been developed. The majority of methods analyse the total fatty acid composition of plasma. It is more difficult to determine the fatty acid composition of a specific class, e.g. phospholipids.

The latter case demands laborious separation processes. The standard procedure is the Folch method, where the samples are extracted with a solvent, the extract is separated on TLC plates and then the separated lipids are scraped off the TLC plate, transesterified and then determined by gas chromatography. In order to provide a high-throughout method, Masood et al. proposed, in Lipids (2008) 43: 171-180, a process supposedly amenable to automation. The process is said to facilitate the laborious working-up of the samples and the transesterification. This is done by treating samples with methanol, acetyl chloride and toluene. The transesterification takes place at 80° C. and the transesterified fatty acids are then analysed in a gas chromatograph. There is no separation of the individual lipids; all the fatty acids present in the sample are analysed by gas chromatography as methyl esters. The hitherto known methods of analysing the fatty acid composition of plasma demand laborious sample preparation, are time-consuming and, because of the associated costs, are unsuitable for mass testing. Moreover, the known methods are unsuitable for determining the fatty acid composition of individual components because analysis of the total fatty acid composition is already laborious. Thus, in the methods known hitherto, the component in which the fatty acid composition was to be determined had to be separated from the remaining components. This presents difficulties in the case of the class of the phospholipids because they are difficult to separate off on account of their amphiphilic properties. Chromatographic processes such as those used hitherto require large amounts of sample on the one hand, and are very laborious on the other. There was therefore a need to provide a method of carrying out the fatty acid composition of one class of lipids directly, without laborious separation processes.

Another problem is that the currently known methods of determination require a relatively large amount of blood. Testing the fatty acid composition is particularly important in children so that developmental disorders due to incorrect nutrition can be detected as early as possible. However, the possibility of repeated testing and serial testing is restricted by the need for a large volume of blood plasma.

Another drawback of the methods known hitherto is that a large quantity of solvents is required to work up the samples, the disadvantages being not only the cost of the solvents, but also the environmental pollution caused by their use.

One object of the present invention was therefore to provide a method of analysing the fatty acid composition of phospholipids, especially phosphoglycerides, which can be carried out in a short time, without laborious sample preparation and with only a small solvent requirement, and which yields reproducible results. Another object of the invention was to provide a method amenable to serial testing and especially automation. Yet another object was to provide a method suitable for small amounts of sample. One particular object of the present invention was to provide a method which allows direct determination of the fatty acids and their composition in phosphoglycerides without laborious separation processes and without requiring large amounts of sample or solvent.

The methods known in the state of the art use either thin layer chromatography or solid phase extraction. Both are laborious, slow and sample-intensive methods that are unsuitable for control testing.

The specific class of the phospholipids or phosphoglycerides is particularly demanding because its emulsifying properties make it difficult to separate from other aqueous or fat-containing components or its variable solution behaviour makes it difficult to separate off. In addition, phosphoglycerides are present in a biological fluid, especially plasma, i.e. a complex matrix consisting of thousands of components, which makes the separation process particularly complicated.

In the state of the art the determination of fatty acid composition required essentially four steps, namely a lipid extraction, e.g. by the standard Folch method, then a lipid class separation by thin layer chromatography or solid phase extraction, then the transesterification of the component obtained in the separation process, and finally a GC analysis of the transesterified fatty acids.

To date it has not been possible to reduce or completely omit the working-up steps described in the state of the art. Rather than separate the phospholipid class from a biological fluid by laborious processes and then determine the fatty acids in this separated phase, the invention strings together three simple steps which are very reliable and allow a non-laborious determination of the fatty acid composition.

The stated objects are achieved by the method according to the invention as defined in the claims. The kit according to the invention further provides the appropriate means of carrying out the method.

The invention provides a method of determining the fatty acid composition of phosphoglycerides wherein methanol is added to a sample containing phosphoglycerides, the combination is mixed, precipitated material is separated from the methanol phase, an alkali metal alkoxide is added to the methanol phase as a base to catalyse a transesterification, and the methyl esters formed in the solution obtained after the transesterification are extracted from the solution and separated by gas chromatography.

The method according to the invention dispenses with the extraction and separation that were necessary in the known methods, by a skilful coupling of already known steps.

This coupling makes it possible specifically to avoid the most laborious steps, namely the lipid extraction and the lipid class separation, and hence also the extraction of large amounts of solvent which have to be used for this purpose.

This is only possible if the steps defined according to the invention are observed. This is done by adding methanol to a sample containing phosphoglycerides and mixing the components, which already precipitates part of the unwanted material and enables it to be separated off. An alkali metal alkoxide is added as a base to the methanol phase, in which the phosphoglycerides are dissolved, and catalyses a transesterification. The methyl esters obtained in this transesterification can then be extracted from the solution and separated by gas chromatography. This simple process saves time, expense, solvent and laborious analysis, but nevertheless yields very reliable results.

It has been found, surprisingly, that specifically methanol offers the ideal solution properties for solving the aforementioned problems. On the one hand proteins are precipitated when using methanol, and on the other hand the non-polar lipids, namely cholesteryl esters and triglycerides, which seriously interfere with the analysis, can also be separated off because they are insoluble, or both sparingly soluble, in the methanol/water mixture. Thus a simple measure at the start has already separated off a large part of the troublesome constituents. In other words a lipid class separation is no longer necessary since methanol dissolves the desired constituents so selectively that work can continue simply with the methanol/water mixture. It is important to use a methanol/water mixture because proteins would dissolve in a pure water phase, while cholesteryl esters and triglycerides would dissolve in a pure methanol phase. Surprisingly, the effect according to the invention is only achieved by mixing methanol with the aqueous sample.

The method according to the invention thus makes it possible, surprisingly, to determine the fatty acid composition of phosphoglycerides in a simple, reproducible manner without laborious sample preparation and without the need for time-consuming separation steps.

Phosphoglycerides or phosphatides are understood here as meaning lipids made up of the basic units phosphoric acid, glycerol and fatty acids, i.e. saturated or unsaturated aliphatic carboxylic acids having up to 30 C atoms and normally 8 to 26 C atoms. The expression “phosphoglyceride” denotes especially glycerol derivatives esterified on two OH groups with fatty acid residues and on the third OH group with phosphoric acid, it being possible for the phosphoric acid residue to be further esterified, e.g. with choline.

Surprisingly, it has been found that the methanol used according to the invention is an ideal reagent for achieving several advantageous objectives. Firstly, methanol separates the desired phosphoglycerides from other constituents contained in the sample, such as proteins and non-polar lipids, because it has a selective dissolving power for phosphoglycerides, but not for other sample constituents that interfere with the test. Methanol dissolves phosphoglycerides almost completely, precipitates proteins present in the plasma and dissolves non-polar lipids only slightly, if at all. Adding methanol is therefore a simple way of increasing the concentration of the desired phosphoglyceride-containing compounds and decreasing the concentration of the unwanted proteins and non-polar lipids. Therefore, in a first stage, methanol is added to the phosphoglyceride-containing sample and the combination is mixed and then centrifuged.

The starting material or sample material for the method according to the invention can be any phosphoglyceride-containing material; it can be either a natural sample or a synthetically prepared mixture (e.g. for comparison purposes). Normally samples of body fluids or body constituents are tested. The phosphoglyceride-containing sample used in the method according to the invention is normally a body fluid, especially blood and preferably blood plasma or blood serum. Plasma denotes a blood sample from which all the cellular constituents have been removed, while blood serum denotes a sample from which not only the cellular constituents but also the clotting factors have been removed. The sample material is preferably blood, especially blood plasma or blood serum.

Plasma contains various lipid fractions: polar components other than phosphoglycerides, such as sphingomyelin and non-esterified fatty acids, and non-polar lipids such as cholesteryl esters and triglycerides. A correlation with the fatty acid composition of cells has been found in phosphoglycerides, which are a group of phospholipids with glycerol as the basic framework. It has been found that this class is less sensitive to short-term changes. Its influence can be investigated by comparing the fatty acid composition.

It has been found that the fatty acid composition of phosphoglycerides is suitable as a marker because it correlates very well with the fatty acid composition of cells.

Thus, after the addition of methanol, a mixture of water and methanol forms (water from the sample and added methanol) which has the solution properties described above. The amount of methanol added is not critical per se. It should not be too large to keep the solvent consumption within economic limits. Also, if the ratio of methanol to water is too high (too much methanol in relation to the water present in the sample), unwanted, less polar substances might be dissolved. Nor should the amount of methanol be too small to be able to contribute the desired solution properties. Also, if the sample contains too little methanol and too much water, the phospholipids might not dissolve completely under certain circumstances. The amount depends inter alia on the water content of the sample and the proportion of constituents to be dissolved. Those skilled in the art can easily find out the appropriate amount by means of routine experiments. It has been found that a suitable volume of methanol corresponds to 3 to 15 times, preferably 5 to 7 times, the sample volume. The methanol is preferably of reagent grade.

To avoid unwanted reactions in the sample; the first step of the method according to the invention is preferably carried out at room temperature or below. Suitable temperatures are in the range from 0 to 45° C., preferably from 0 to 25° C. and particularly preferably from 5 to 15° C. A suitable procedure is to add cooled methanol, i.e. methanol at a temperature ranging from 0 to 20° C., preferably from 5 to 20° C., to the sample at either body temperature, room temperature or refrigerator temperature, so that the temperature of the mixture is then within appropriate limits.

As explained above, the effect of adding methanol is to precipitate proteins and any other ingredients insoluble in methanol. The precipitated constituents can be separated from the mixture by using methods known per se. Preferably, separation is effected by centrifuging the mixture. The centrifugation can be performed in a manner known per se until the cellular constituents have settled at the bottom of the centrifuge vessel. The centrifugation conditions are those conventionally used for such separations. Thus, a suitable procedure is to centrifuge at ca. 500 to 1500×g. A centrifugation time of 1 to 10 min, preferably of 3 to 7 min, has proved particularly suitable.

It is also possible to use other methods of separating off the precipitated constituents, e.g. filtration etc.

The liquid obtained in the separation, i.e. the methanol phase, which is normally the supernatant in the case of centrifugation, is passed on to the next stage. The supernatant contains the desired phosphoglycerides together with sphingomyelin and non-esterified fatty acids, as well as a small proportion of non-polar lipids. In a second stage the methanol phase, which contains an increased concentration of phosphoglycerides along with other polar and non-polar constituents, is subjected to a transesterification. This is carried out in order to convert the fatty acids to derivatives which can be separated by gas chromatography, i.e. which are volatile in the range appropriate for gas chromatography.

Methyl esters are normally prepared for this purpose. Other esters suitable for gas chromatography can also be prepared, in which case the appropriate alcohol is added.

Fatty acid methyl esters are volatile in a range that is particularly appropriate for gas chromatographic separation.

The transesterification to form methyl esters takes place in the supernatant solution, which already contains the esterifying agent, i.e. methanol. A basic catalyst is used to carry out the transesterification. It has been found that the use of an alkali metal alkoxide as the basic catalyst specifically effects the transesterification of the fatty acids present in the phosphoglycerides, but not the transesterification of the free fatty acids, or the fatty acids bound to sphingomyelin and cholesteryl esters, that are also present in the solution. By virtue of this selectivity in the transesterification, it is possible specifically to convert the desired fatty acids, i.e. those bound as phosphoglycerides, to methyl esters without at the same time esterifying the unwanted, free fatty acids.

It has been found that alkali metal alkoxides are particularly suitable catalysts, it being preferable to use sodium or potassium, especially sodium, as the alkali metal. The alkoxide can be derived from an alcohol having 1 to 3 carbon atoms, especially methanol. It is particularly preferable to use sodium methoxide as the basic catalyst.

To keep the esterification as selective as possible, it has proved appropriate to carry out the esterification reaction at a temperature ranging from 0 to 45° C., preferably from 15 to 30° C., and particularly preferably at room temperature, i.e. in the range from 20 to 25° C.

It has also been found that the transesterification reaction proceeds very rapidly, so it can be stopped after a short time in order to extract the methyl esters. A reaction time ranging up to 10 min is already sufficient to convert substantially all the fatty acids bound in the phosphoglycerides into methyl esters. Preferably, therefore, the reaction can be stopped after 2 to 10 min by adding an acid, preferably a methanolic acid. It has been found that a longer reaction time is not detrimental because the transesterification reaction is greatly preferred over the saponification reaction. However, to increase the efficiency, the transesterification reaction should not be carried out for longer than 10 min.

In a third stage the fatty acid esters formed from the phosphoglyceride fatty acids are then extracted into an organic solvent and subjected to a gas chromatographic separation. The extract can be directly injected. A suitable organic solvent is any solvent capable of extracting the fatty acid methyl esters from the methanol phase. Normally a suitable solvent is a non-polar solvent that is inert towards the reactants, or a mixture of solvents. The organic solvent must also be suitable for injection into the gas chromatograph, i.e. must not produce a signal at an inappropriate time. Such solvents are well known to those skilled in the art. Hydrocarbons, especially hexane, have proved particularly suitable.

The extraction into an organic solvent can be repeated in order to increase accuracy; a second extraction has proved beneficial. Furthermore, the solvent can be evaporated off after the extraction so that the sample can then be taken up in a defined amount of solvent for the gas chromatography.

To avoid changes in the fatty acids, especially the oxidation of unsaturated bonds, during the work-up, an antioxidant can be added to the work-up mixture. Antioxidants for preventing fatty acid oxidation are well known to those skilled in the art, an example being BHT.

It is possible according to the invention to increase the accuracy and reproducibility of the determination even further by using internal standards. As is familiar to those skilled in the art, the internal standard is tested together with the sample and allows conclusions to be drawn about the recovery of the compounds of interest. On the basis of the values obtained, it is then possible to calculate the actual concentration. The internal standard used is preferably at least one phosphoglyceride compound or a mixture of phosphoglyceride compounds, particularly preferably at least one di-fatty acid-sn-glycero-3-phosphocholine, the fatty acid(s) used preferably having 14 to 20 C atoms.

All the documents of the state of the art have assumed that the fatty acid composition of phospholipids or phosphoglycerides can only be determined if the phospholipids have first been separated from other lipid constituents. Surprisingly, it has now been found that such a separation process, which is time-consuming and requires a high consumption of solvent, is not necessary if the individual steps of the method according to the invention are observed.

The method according to the invention makes it possible to analyse the fatty acid composition of phosphoglycerides in a simple manner and to detect all the fatty acids present, qualitatively and quantitatively. The reproducibility is high and can be further increased by using suitable standards. The results obtained by the method according to the invention therefore enable reliable conclusions to be drawn. The doctor is thus equipped with a means of analysing various types of disorder and disease arising due to an inadequate fatty acid supply or unbalanced diet, and to monitor them during the treatment.

The method according to the invention is suitable both for single analyses and especially for serial tests and large clinical studies.

The method according to the invention is suitable both for single analyses and especially for serial tests and large clinical studies. Hitherto the fatty acid composition of plasma has not been tested on larger groups because the analysis was far too laborious. The method according to the invention makes it possible for the first time to carry out larger-scale studies.

With the methods known hitherto, an experienced laboratory technician required two working days for ten samples, whereas the method according to the invention makes it possible to analyse more than 50 samples per day, or more. In other words the effort is reduced by a factor of 10. The proportion of solvent is simultaneously reduced by more than 90%.

In summary, the method according to the invention is distinguished by considerable time and cost savings made possible by separation of the unwanted constituents, namely protein and cholesteryl esters and triglycerides, by precipitation with methanol, and transesterification under specific conditions.

The invention also provides a kit for analysing the fatty acid composition of phosphoglycerides in plasma, said kit providing all the items needed for the analysis. The kit comprises a sample receptacle, methanol for dissolving the phosphoglycerides, an alkali metal alkoxide as transesterification catalyst, optionally a methanolic acid as stopper, an organic solvent for extracting the esterified fatty acids and as carrier for the gas chromatography, and at least one phosphoglyceride, preferably at least one di-fatty acid-sn-glycero-3-phosphocholine, as internal standard.

The reagents are each in separate containers and can be apportioned as defined by the method according to the invention.

In one preferred embodiment, the alkali metal alkoxide is sodium methoxide.

The internal standard used can be a single compound or a mixture of different phosphoglycerides, it being possible to distinguish between different compounds in the fatty acids and/or in another ester bound to the phosphoric acid residue. Conventionally the internal standard used consists of compounds which are expected in the test sample or are similar to the expected compounds. It is particularly preferable to use a phosphocholine esterified with saturated C15 and/or C17 alkanoic acids, because this fatty acid does not occur in the sample but is very similar to the expected ones (C16/C18). In other words it is preferable to choose a compound which is strongly represented in the sample, e.g. phosphocholine, but which carries a fatty acid that is only weakly represented in the sample, if at all.

As explained above, the organic solvent can be a single compound or a mixture. It is preferably a hydrocarbon, especially hexane.

Furthermore, in one preferred embodiment, the kit additionally contains a stopper. This is preferably an acid, especially an acid dissolved in methanol. A methanolic mineral acid, such as methanolic hydrochloride, has proved particularly suitable.

In one preferred embodiment, the reagents are already provided in the proportions required for carrying out the method. For this purpose the methanol is provided in an amount corresponding to 5 to 20 times the amount of sample, the alkali metal alkoxide is provided in an amount corresponding to 0.1 to 0.5 times the volume of sample, the internal standard solution is provided in a volume corresponding to 0.8 to 1.2 times the volume of sample, and the extractant is provided in a volume corresponding to 2 to 5 times the volume of sample.

Particularly preferably, the kit contains methanol/alkali metal oxide/organic solvent in a ratio of 5-10:0.05-0.2:2-10.

The method according to the invention makes it possible to introduce the fatty acid composition of plasma phosphoglycerides as a novel biomarker. The invention therefore also provides the use of the analysis of the fatty acid composition of plasma phosphoglycerides as a biological marker for monitoring the mental development of children and the elderly.

An advantage of testing the phospholipids is that their composition is influenced less by food intake than e.g. the composition of the triglycerides. If additionally the sphingomyelin is separated from the phospholipids to leave the phosphoglyceride group, the determination is refined further and opens up more analysis options. To date, separation of the sphingomyelin from the phospholipids has not been a practical possibility because the methods used did not allow this separation. However, more than 95% of sphingomyelin consists of saturated and monounsaturated fatty acids, which are of lesser significance physiologically and analytically. Separation of this non-evidential constituent therefore affords an even more accurate diagnosis and markedly increases the sensitivity.

The method according to the invention makes it possible specifically to test only the phosphoglycerides, which contain a particularly high proportion of polyunsaturated fatty acids that are of particular value for the diagnosis, so the method according to the invention enables one to concentrate on the diagnosis of linoleic acid and a-linolenic acid and their metabolites, such as arachidonic acid and docosahexaenoic acid, which play an important role in metabolism.

### FIGURES

FIG. 1 shows the composition of the main plasma lipid classes in percent (PL=phospholipids, NEFA=non-esterified fatty acids, TAG=triacylglycerols and CE=cholesteryl esters) in the Folch extracts (Diagram A) and in the methanolic supernatants of the 16 different samples (Diagram B).

FIGS. 2 and 3 shows correlation curves for the concentrations of the fatty acids and of docosahexaenoic acid in phosphoglycerides, as examples demonstrating the high correlation over a broad concentration range.

The Examples which follow illustrate the method according to the invention in greater detail, but are not to be regarded as implying a limitation.

### EXAMPLE 1

### Reagents and Samples

Analytical-grade solvents were obtained from Merck KGAA (Darmstadt, Germany). Methanolic HCl (3 N) and sodium methoxide (25 wt. % in methanol) were acquired from Sigma-Aldrich (Taufkirchen, Germany). Two internal standards were used. Internal standard A was prepared by dissolving pentadecanoic acid, cholesteryl pentadecanoate, tripentadecanoin and 1,2-dipentadecanoyl-sn-glycero-3-phosphocholine (Sigma-Aldrich) in methanol/chloroform (35:15). Internal standard B was made up of 1,2-dipentadecanoyl-sn-glycero-3-phosphocholine dissolved in methanol. For determination of the efficiency of the base-catalysed transesterification, octadecane (Sigma-Aldrich) was dissolved in methanol as an internal standard. To prevent oxidation of the fatty acids, 2 g/l of 2,6-di-tert-butyl-p-cresol (butylated hydroxytoluene, BHT, Sigma-Aldrich) were added to each internal standard. GLC-85, containing 32 fatty acid methyl esters (Nu-Check Prep, Inc., Elysian, Minn., USA), was used as an external standard. L-α-phosphatidylcholine (type XVI-E, approx. 99% TLC) and sphingomyelin (chicken egg yolk, ≧98% TLC) were acquired from Sigma-Aldrich. A mixture of sodium carbonate, sodium, hydrogen carbonate and sodium sulfate (1:2:2, Merck KGAA) was used as a buffer for neutralization after the acid-catalysed transesterification. 33 blood samples from healthy volunteers (fasted or non-fasted) were collected in Vacutainer tubes containing EDTA. The plasma was separated off by centrifugation (900×g, 5 min) and stored at −20° C. until required for analysis.

### Folch Extraction (Comparison)

100 μl of internal standard A were added to 250 μl of plasma; the lipids were extracted by a modified Folch method (J. Folch, M. Lees and G. H. Sloane Stanley (1975): A simple method for the isolation and purification of total lipids from animal tissues, J. Biol. Chem. 226, 497-509) using chloroform/methanol (2:1, v/v) and washed twice with NaCl solution (2% in water). The extracts were dried at 30° C. under reduced pressure and taken up in 400 μl of chloroform/methanol (1:1) for application to thin layer chromatography plates.

### Lipid Fraction Separation by TLC, Acid-Catalysed Transesterification

N-heptane, diisopropyl ether and acetic acid (60:40:3) were used as the mobile phase to separate the phospholipids from free cholesterol, non-esterified fatty acids, triacylglycerols and cholesterol esters. The corresponding bands were scraped off the TLC plate and transferred to glass tubes, and 1.5 ml of methanolic HCl were added. The sealed tubes were shaken for 30 sec and heated at 85° C. for 45 min. After cooling to room temperature, the samples were neutralized with carbonate buffer. 1 ml of hexane was added for methyl ester extraction. After centrifugation for 5 minutes at 900×g, the upper hexane phase was transferred to another glass tube. The extraction was repeated and the combined extracts were dried to dryness under a stream of nitrogen at room temperature. The dry residue was taken up in 50 μl of hexane (containing 2 g/l of BHT) for gas chromatographic (GC) analysis.

### Base-Catalysed Transesterification of the Phosphoglyceride Fatty Acids (According to the Invention)

For analysis of the plasma phosphoglyceride fatty acids, 100 μl of plasma, 100 μl of internal standard B and 0.6 ml of methanol (precooled to 5° C.) were combined in glass tubes and shaken for 30 sec. After centrifugation for 5 minutes at 900×g, the supernatant was transferred to another glass tube. After the addition of 25 μl of sodium methoxide solution, the tubes were shaken and the synthesis of the methyl esters was continued at room temperature. The reaction was stopped after 3 min by adding 75 μl of methanolic HCl. 300 μl of hexane were added for extraction and the tubes were shaken for 30 sec. The upper hexane phase was transferred to a 2 ml glass tube. The extraction was repeated and the combined extracts were dried under a stream of nitrogen at room temperature. The dry residue was taken up in 50 μl of hexane (containing 2 g/l of BHT) for GC analysis.

To evaluate the lipid compositions in the methanolic supernatant after precipitation of the protein in the plasma, and to compare the recovery of the phospholipids in the methanolic supernatant with the Folch extract, the supernatant was applied to a TLC plate. The lipids were separated by TLC and converted to fatty acid methyl esters (FAME) by acid-catalysed transesterification.

The base-catalysed transesterification and the extraction of the fatty acid methyl esters were optimized by using a model sample, 100 μl of water (representing plasma), 100 μl of internal standard B and 100 μl of octadecane standard (which did not participate in the reactions). The ratio of the peak areas of methyl pentadecanoate to octadecane was used as an indicator of the efficiency of the transesterification or extraction.

### Chromatography

The individual FAME were quantitatively evaluated by gas chromatography with a flame ionization detector. The GC analysis was performed with a BTX-70 column (60 m×0.32 mm, SGE, Weiterstadt, Germany) using an Agilent 5890 Series II gas chromatograph (Agilent, Waldbronn, Germany). Identical GC conditions (initial temperature: 130° C., rate of increase: 3° C./min to 170° C., 1.5° C./min to 180° C. and 3° C./min to 210° C., isothermal period: 23 min, carrier gas: He, column head pressure: 1.5 bar) were used for all the gas chromatographic analyses.

### Quantitative Data Evaluation

Individual FAME were identified by comparison with authentic standards. The proportion of each fatty acid methyl ester relative to methyl pentadecanoate (internal standard) was determined using GLC-85 as external standard. EZChrom Elite Version 3.1.7 was used for the peak integration.

### Statistical Analysis

For fatty acids with a length of between 14 and 24 carbon atoms, the results were expressed as absolute plasma concentrations (mg/l) and as percentages (percent by weight). The FA data were presented as mean±SD. The correlations were evaluated using the two-sided Spearman test and paired t-tests for comparing means (P less than 0.05 was regarded as statistically significant). The statistical analysis was performed with SPSS for Windows, Version 15.0.1 (SPSS Inc., Chicago, Ill., USA).

### Results

### Analysis of the Phosphoglyceride Fatty Acids

The intra-assay reproducibility (n=8) of the phospholipid analysis was determined by Folch extraction/TLC in comparison with the results obtained by protein precipitation with methanol/TLC and in comparison with the results obtained from phosphoglycerides by the base-catalysed transesterification according to the invention. The phospholipid fatty acid concentrations (mg/l) and compositions (percent by weight) were comparable in Folch extracts and methanolic supernatants (Table 1 and Table 2), but exhibited statistically significant differences for some fatty acids. As expected, the phosphoglyceride fatty acid concentrations differed from those in phospholipids. The concentrations of the saturated fatty acids C20: 0, C22: 0 and C24: 0 and of the monounsaturated fatty acid C24: 1 n-9 in the phosphoglycerides were below the detection limit. The total phosphoglyceride FA concentration was about 10% lower than in phospholipids, although some individual fatty acids exhibited higher concentrations. For phosphoglycerides obtained by base-catalysed transesterification, the CV for all the FA was found to be below 4%; C18: 3 n-3, which makes up 0.21% of the total fatty acids, had the highest CV (3.8%).

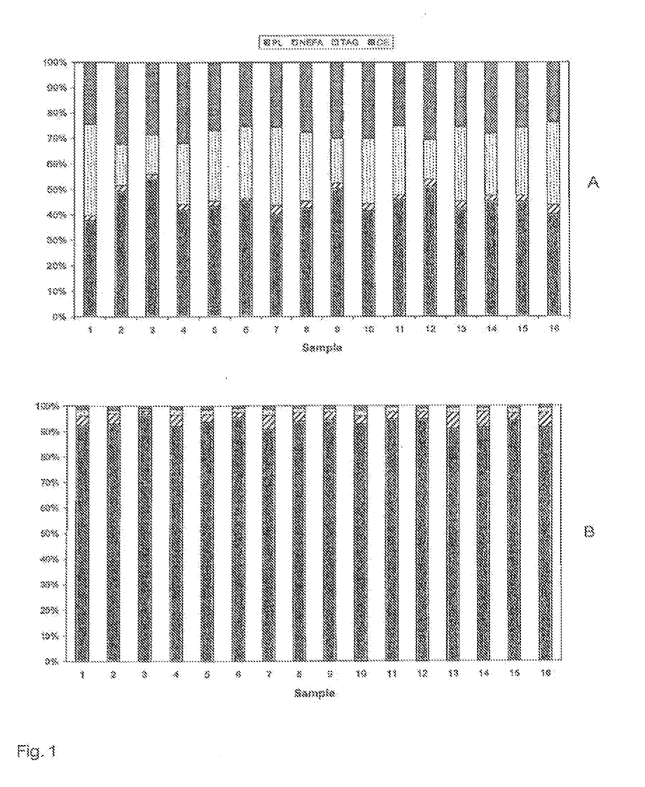

Sixteen different plasma samples were used to establish the relationship between the fatty acid concentration obtained for plasma phospholipids by extraction/TLC and acid-catalysed transesterification, and the concentration after base-catalysed transesterification of the methanolic supernatant of the plasma protein precipitate. The plasma lipid composition of the sample was estimated from the sum of the fatty acids determined in the individual fractions after extraction and TLC separation. The phospholipids made up 37.7% to 54.6%, the non-esterified fatty acids 1.3% to 3.7%, the triacylglycerols 15.4% to 35.8% and the cholesteryl esters 23.6% to 32.4% (FIG. 1A). The lipid composition of the methanolic supernatant after protein precipitation and TLC was 90.9% to 96.8% of phospholipids, 1.3% to 6.3% of non-esterified fatty acids, 0.9% to 2.5% of triacylglycerols and 0.8% to 2.0% of cholesteryl esters (FIG. 1B). Non-esterified fatty acids, sphingomyelin fatty acids and cholesteryl fatty acids were not converted to FAME by reaction with sodium methoxide under the indicated conditions. The total phospholipid FA concentration for these samples was on average 1317.4 mg/l (1054.2 mg/l to 1908.3 mg/l), depending on the extraction method. A total FA content of 1229.9 mg/l (970.4 mg/l to 1836.3 mg/l) was found in plasma phosphoglycerides for the base-catalysed transesterification.

The correlation of the FA concentrations and the percentage contributions to the phospholipids obtained by Folch extraction/TLC and protein precipitation with methanol/TLC, and in phosphoglycerides obtained by base-catalysed transesterification, was determined (Table 3). For the concentrations of all the analysed fatty acids in phospholipids obtained by extraction/TLC, and in phosphoglycerides obtained by base-catalysed transesterification, correlation coefficients of more than 0.9 (P<0.0001) were achieved (except for C14: 0 and C18: 3 n-6). Both C14: 0 and C18: 3 n-6 exhibited very low concentrations and their proportion of the total fatty acids in phospholipids and phosphoglycerides was below 1%. For the percentage of all the analysed fatty acids in phospholipids obtained by Folch extraction/TLC, and in phosphoglycerides obtained by base-catalysed transesterification, the correlation coefficients were higher than 0.9 (P<0.0001) for most of the fatty acids. Only for C14: 0, C20: 1 n-9, C22: 4 n-9 and C18: 3 n-3 were the r values between 0.76 and 0.89 with P values of ≦0.001.

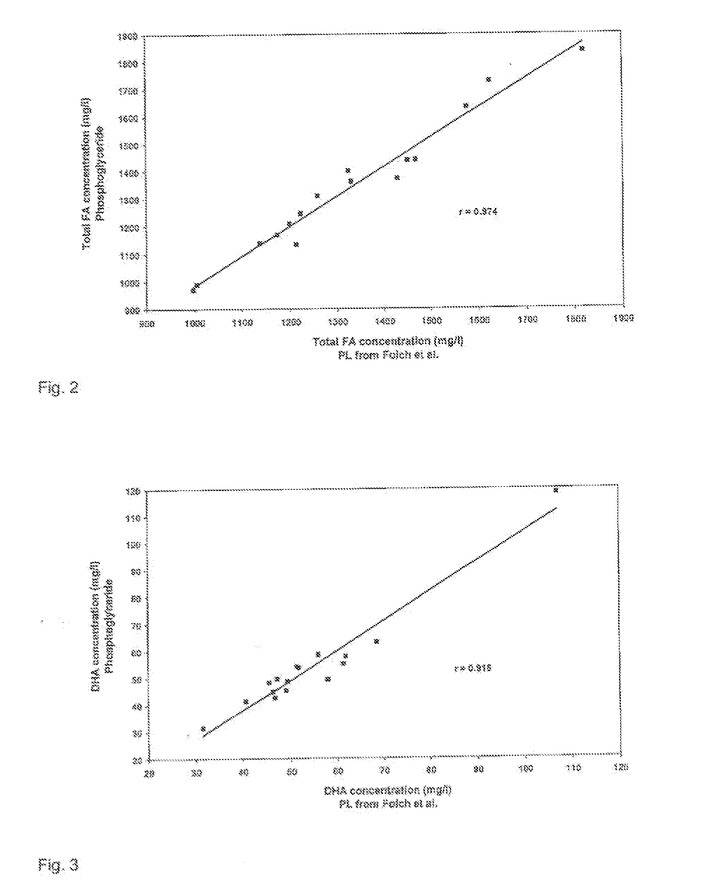

FIGS. 2 and 3 show the correlation curves for the fatty acids from phosphoglycerides by way of comparison.

The recovery of the phospholipids (n=16) in the methanolic supernatants was found to be 88.1%±6.6% (mean±SD) compared with Folch extraction. The internal standard, which was added directly to the plasma, made it possible to correct for the loss of phospholipids, so 101.0%±2.6% of the phospholipids was correctly determined in the methanolic supernatants.

As hydrolysis of the methyl esters might be a problem if water (from the plasma sample) is present during the base-catalysed transesterification, the reaction yields were examined in methanolic solution containing 100 μl of water and 100 μl of internal standard B. It was found that reaction times of between 3 min and 10 min assured a complete transesterification of the fatty acids from the phosphoglycerides. The recovery of internal standard B was 99.1%±0.8% (mean±SD), based on the octadecane standard, in 8 independent analyses.

After the base-catalysed transesterification the FAME were extracted twice with 300 μl of hexane. To evaluate the extraction efficiency, the samples were re-extracted with 1 ml of hexane. These extracts contained less than 1% of the total FAME which had been obtained by the previous extractions.

Storage of the GC-ready derivatives for one month at −20° C. showed no significant changes in the FA concentrations.

The Tables which follow show:

### Table 1

Intra-assay (n=8) reproducibility of the fatty acid concentrations (mg/l) in phospholipids (FL) obtained by Folch extraction/TLC and protein precipitation with methanol/TLC, and in phosphoglycerides obtained by base-catalysed transesterification

### Table 2

Intra-assay (n=8) reproducibility of the fatty acid composition (%) of phospholipids (PL) obtained by Folch extraction/TLC and protein precipitation with methanol/TLC, and of phosphoglycerides obtained by base-catalysed transesterification

### Table 3

Correlations (n=16, P<0.0001, except for *P=0.001) of the fatty acid concentrations (mg/l) and compositions (% by weight) in phospholipids (PL) by Folch extraction/TLC and protein precipitation with methanol/TLC, and in phosphoglycerides obtained by base-catalysed transesterification

TABLE 1

| | PL | | | | | |
|---|---|---|---|---|---|---|
| | from Folch et al. | | PL in methanol | | Phosphoglycerides | |
| | | Coefficient | | Coefficient | | Coefficient |
| Fatty acids (FA) | Mean | of variation | Mean | of variation | Mean | of variation |
| Saturated FA | | | | | | |
| C14:0 | 5.24 | 2.4 | 6.38 | 3.6 | 7.95 | 3.2 |
| C16:0 | 368.36 | 0.8 | 376.36 | 1.2 | 354.78 | 0.7 |
| C17:0 | 5.37 | 1.3 | 5.41 | 2.5 | 4.93 | 1.4 |
| C18:0 | 185.00 | 1.5 | 188.00 | 2.0 | 168.46 | 1.1 |
| C20:0 | 6.67 | 1.4 | 7.06 | 3.2 | ND | ND |
| C22:0 | 15.64 | 1.5 | 16.35 | 1.5 | ND | ND |
| C24:0 | 14.11 | 2.7 | 14.94 | 2.9 | ND | ND |
| Monounsaturated FA | | | | | | |
| C14:1 | ND | ND | ND | ND | ND | ND |
| C16:1n-7 | 9.85 | 1.5 | 10.55 | 1.7 | 14.05 | 2.0 |
| C18:1n-7 | 19.88 | 1.4 | 19.64 | 1.5 | 20.69 | 1.0 |
| C18:1n-9 | 143.35 | 1.0 | 142.99 | 1.4 | 156.59 | 1.3 |
| C20:1n-9 | 2.35 | 1.5 | 2.18 | 3.3 | 2.26 | 2.3 |
| C24:1n-9 | 28.89 | 1.2 | 29.63 | 1.6 | ND | ND |
| n-9 PUFA | | | | | | |
| C20:3n-9 | 2.64 | 2.9 | 2.79 | 4.9 | 2.76 | 1.9 |
| n-6 PUFA | | | | | | |
| C18:2n-6 | 248.15 | 1.1 | 243.21 | 1.4 | 251.05 | 1.4 |
| C18:3n-6 | 1.73 | 8.0 | 1.84 | 2.8 | 2.15 | 2.5 |
| C20:2n-6 | 3.78 | 1.7 | 3.85 | 3.9 | 3.96 | 1.9 |
| C20:3n-6 | 43.25 | 1.3 | 40.91 | 1.7 | 41.59 | 1.3 |
| C20:4n-6 | 133.09 | 1.2 | 126.76 | 1.4 | 124.89 | 1.3 |
| C22:4n-6 | 5.41 | 1.5 | 5.20 | 5.1 | 4.64 | 2.6 |
| C22:5n-6 | 4.08 | 3.2 | 3.98 | 2.9 | 3.70 | 2.5 |
| n-3 PUFA | | | | | | |
| C18:3n-3 | 1.92 | 2.5 | 1.74 | 3.0 | 2.59 | 3.8 |
| C20:5n-3 | 10.50 | 1.3 | 9.74 | 1.6 | 9.99 | 1.6 |
| C22:5n-3 | 12.00 | 1.1 | 10.68 | 1.2 | 10.69 | 1.5 |
| C22:6n-3 | 46.22 | 1.9 | 41.13 | 0.8 | 42.15 | 1.4 |
| Total fatty acids | 1317.42 | 0.9 | 1311.29 | 1.3 | 1229.85 | 0.9 |

TABLE 2

| | PL | | | | | |
|---|---|---|---|---|---|---|
| | from Folch et al. | | PL in methanol | | Phosphoglycerides | |
| | | Coefficient | | Coefficient | | Coefficient |
| Fatty acids (FA) | Mean | of variation | Mean | of variation | Mean | of variation |
| Saturated FA | | | | | | |
| C14:0 | 0.40 | 3.0 | 0.49 | 3.9 | 0.65 | 3.4 |
| C16:0 | 27.96 | 0.3 | 28.70 | 0.2 | 28.85 | 0.6 |
| C17:0 | 0.41 | 0.7 | 0.41 | 1.8 | 0.40 | 0.6 |
| C18:0 | 14.04 | 0.9 | 14.34 | 1.4 | 13.70 | 0.7 |
| C20:0 | 0.51 | 1.1 | 0.54 | 1.9 | ND | ND |
| C22:0 | 1.19 | 1.4 | 1.25 | 0.4 | ND | ND |
| C24:0 | 1.07 | 2.6 | 1.14 | 2.7 | ND | ND |
| Monounsaturated FA | | | | | | |
| C14:1 | ND | ND | ND | ND | ND | ND |
| C16:1n-7 | 0.75 | 1.7 | 0.80 | 1.5 | 1.14 | 1.8 |
| C18:1n-7 | 1.51 | 0.6 | 1.50 | 1.3 | 1.68 | 0.6 |
| C18:1n-9 | 10.88 | 0.5 | 10.90 | 0.7 | 12.73 | 0.7 |
| C20:1n-9 | 0.18 | 1.6 | 0.17 | 3.1 | 0.18 | 2.6 |
| C24:1n-9 | 2.19 | 0.8 | 2.26 | 0.9 | ND | ND |
| n-9 PUFA | | | | | | |
| C20:3n-9 | 0.20 | 2.3 | 0.21 | 3.7 | 0.22 | 1.8 |
| n-6 PUFA | | | | | | |
| C18:2n-6 | 18.84 | 0.8 | 18.55 | 0.3 | 20.41 | 0.7 |
| C18:3n-6 | 0.13 | 8.5 | 0.14 | 3.6 | 0.17 | 2.4 |
| C20:2n-6 | 0.29 | 1.5 | 0.29 | 3.0 | 0.32 | 1.3 |
| C20:3n-6 | 3.28 | 0.6 | 3.12 | 0.6 | 3.38 | 0.7 |
| C20:4n-6 | 10.10 | 0.4 | 9.67 | 0.4 | 10.15 | 0.8 |
| C22:4n-6 | 0.41 | 1.4 | 0.40 | 4.4 | 0.38 | 2.0 |
| C22:5n-6 | 0.31 | 3.2 | 0.30 | 2.6 | 0.30 | 2.1 |
| n-3 PUFA | | | | | | |
| C18:3n-3 | 0.15 | 2.1 | 0.13 | 2.3 | 0.21 | 3.7 |
| C20:5n-3 | 0.80 | 0.6 | 0.74 | 0.6 | 0.81 | 0.7 |
| C22:5n-3 | 0.91 | 0.6 | 0.81 | 1.0 | 0.87 | 0.9 |
| C22:6n-3 | 3.51 | 1.1 | 3.14 | 0.7 | 3.43 | 0.9 |

TABLE 3

| | Folch PL vs. | | | |
|---|---|---|---|---|
| | methanol PL | | Folch PL vs. | |
| | FA | FA | phosphoglycerides | |
| | concen- | compo- | FA | FA |
| Fatty acids (FA) | tration | sition | concentration | composition |
| Saturated FA | | | | |
| C14:0 | 0.974 | 0.941 | 0.841 | 0.835 |
| C16:0 | 0.994 | 0.900 | 1.000 | 0.918 |
| C17:0 | 0.973 | 0.964 | 0.958 | 0.956 |
| C18:0 | 0.971 | 0.994 | 0.933 | 0.985 |
| C20:0 | 0.906 | 0.979 | ND | ND |
| C22:0 | 0.921 | 0.985 | ND | ND |
| C24:0 | 0.968 | 0.981 | ND | ND |
| Monounsaturated FA | | | | |
| C14:1 | ND | ND | ND | ND |
| C16:1n-7 | 0.929 | 0.958 | 0.924 | 0.962 |
| C18:1n-7 | 0.959 | 0.982 | 0.974 | 0.970 |
| C18:1n-9 | 0.974 | 0.956 | 0.976 | 0.968 |
| C20:1n-9 | 0.956 | 0.909 | 0.913 | 0.804 |
| C24:1n-9 | 0.960 | 0.981 | ND | ND |
| n-9 PUFA | | | | |
| C20:3n-9 | 0.979 | 0.979 | 0.978 | 0.999 |
| n-6 PUFA | | | | |
| C18:2n-6 | 0.979 | 0.991 | 0.974 | 0.956 |
| C18:3n-6 | 0.802 | 0.730* | 0.885 | 0.938 |
| C20:2n-6 | 0.986 | 0.967 | 0.975 | 0.963 |
| C20:3n-6 | 0.991 | 0.990 | 0.974 | 0.982 |
| C20:4n-6 | 0.977 | 0.985 | 0.944 | 0.950 |
| C22:4n-6 | 0.954 | 0.921 | 0.915 | 0.760* |
| C22:5n-6 | 0.987 | 0.984 | 0.979 | 0.996 |
| n-3 PUFA | | | | |
| C18:3n-3 | 0.982 | 0.982 | 0.916 | 0.888 |
| C20:5n-3 | 0.991 | 0.985 | 0.995 | 0.985 |
| C22:5n-3 | 0.952 | 0.964 | 0.950 | 0.940 |
| C22:6n-3 | 0.941 | 0.951 | 0.915 | 0.946 |
| Total fatty acids | 0.988 | | 0.974 | |

1. Method of determining the fatty acid composition of phosphoglycerides wherein methanol is added to a sample containing phosphoglycerides, the combination is mixed, precipitated material is separated from the methanol phase, an alkali metal alkoxide is added to the methanol phase as a base to catalyse a transesterification, and the methyl esters formed in the solution obtained after the transesterification are extracted from the solution and separated by gas chromatography.

2. Method according to claim 1, characterized in that the sample containing phosphoglycerides is a blood sample, especially blood plasma or blood serum.

3. Method according to claim 1, characterized in that sodium methoxide is used as the base.

4. Method according to claim 1, characterized in that the transesterification is carried out at a temperature ranging from 0 to 45° C.

5. Method according to claim 1, characterized in that the transesterification is carried out at a temperature ranging from 15 to 30° C., preferably at room temperature in the range from 20 to 25° C.

6. Method according to claim 1, characterized in that the extraction is carried out with a non-polar solvent.

7. Method according to claim 1, characterized in that the extraction is carried out with hexane.

8. Kit for analysing the fatty acid composition of phosphoglycerides, comprising
a sample receptacle
and, in separate containers,
methanol for dissolving the phosphoglycerides
an alkali metal alkoxide as transesterification catalyst
an organic solvent for extracting the fatty acid methyl esters and as carrier for the gas chromatography
and at least one phosphoglyceride as internal standard.

9. Kit according to claim 8, characterized in that the alkali metal alkoxide is sodium methoxide.

10. Kit according to claim 8, characterized in that it also contains an alcoholic acid as stopper.

11. Kit according to claim 8, characterized in that it contains at least one di-fatty acid-sn-glycero-3-phosphocholine as a standard.

12. Method of determining the fatty acid status of tissue as a biological marker for the mental development of children and for heart diseases, cancer and autoimmune diseases, wherein the fatty acid composition of phospholipids, especially phosphoglycerides, is determined by a method according to claim 1.

13. Method of determining the fatty acid status of tissue as a biological marker for the mental development of children and for heart diseases, cancer and autoimmune diseases, wherein the fatty acid composition of phospholipids, especially phosphoglycerides, is determined by a method according to claim 2.

14. Method of determining the fatty acid status of tissue as a biological marker for the mental development of children and for heart diseases, cancer and autoimmune diseases, wherein the fatty acid composition of phospholipids, especially phosphoglycerides, is determined by a method according to claim 3.

15. Method of determining the fatty acid status of tissue as a biological marker for the mental development of children and for heart diseases, cancer and autoimmune diseases, wherein the fatty acid composition of phospholipids, especially phosphoglycerides, is determined by a method according to claim 4.

16. Method of determining the fatty acid status of tissue as a biological marker for the mental development of children and for heart diseases, cancer and autoimmune diseases, wherein the fatty acid composition of phospholipids, especially phosphoglycerides, is determined by a method according to claim 5.

17. Method of determining the fatty acid status of tissue as a biological marker for the mental development of children and for heart diseases, cancer and autoimmune diseases, wherein the fatty acid composition of phospholipids, especially phosphoglycerides, is determined by a method according to claim 6.

18. Method of determining the fatty acid status of tissue as a biological marker for the mental development of children and for heart diseases, cancer and autoimmune diseases, wherein the fatty acid composition of phospholipids, especially phosphoglycerides, is determined by a method according to claim 7.
